(19) United States
(12) Patent Application Publication
(10) Pub. No.: US 2025/0367088 A1
(43) Pub. Date: Dec. 4, 2025

(54) COMPOSITIONS AND METHODS FOR CARING FOR KERATIN MATERIALS

(71) Applicant: L'Oreal, Paris (FR)

(72) Inventors: Shilpa ARORA, South Planfield, NJ (US); Zavvia RAMOS, Jersey City, NJ (US); Aziza Khader SULEIMAN, Paterson, NJ (US); Anand Ramchandra MAHADESHWAR, Westfield, NJ (US)

(21) Appl. No.: 18/731,085

(22) Filed: May 31, 2024

(51) Int. Cl. A61K 8/42; A61K 8/23; A61K 8/368; A61K 8/49; A61K 8/63; A61K 8/96; A61K 8/9789; A61Q 5/00; A61Q 5/12

(52) U.S. Cl. CPC A61K 8/42; A61K 8/23; A61K 8/368; A61K 8/4926; A61K 8/4946; A61K 8/498; A61K 8/63; A61K 8/965; A61K 8/9789; A61Q 5/006; A61Q 5/12

(57) ABSTRACT

The disclosure relates to compositions having (a) at least one cationic surfactant, (b) at least one benefit agent, (c) optionally at least one anti-dandruff agent, and (d) water, as well as to methods of using the compositions for caring for keratin materials, such as the hair and scalp.

### TECHNICAL FIELD

The disclosure relates to compositions for caring for keratin materials, especially the hair, skin, and/or the scalp, and to methods of using the compositions.

### BACKGROUND

The appearance and/or condition of hair may be affected by both extrinsic and intrinsic factors. For example, exposure to environmental conditions such as ultraviolet radiation from the sun; chemical agents such as those found in shampoos, hair bleaches, hair relaxers, hair dyes, and permanent waving compositions; heat such as curling or straightening irons or hair dryers; and/or mechanical stress such as from brushing or grooming activities, can negatively impact the condition of the hair fibers and scalp. In addition, the condition of the hair and scalp can diminish over time as a result of the natural aging process and/or due to factors such as natural greasiness, sweat, shedded skin cells from the scalp, and other conditions.

Dandruff, for example, is a scalp condition that is experienced by millions of people. Besides the negative impacts on the appearance and/or condition of the individual's hair and/or scalp, dandruff can also have social implications due to a perceived stigma associated with this condition. The occurrence of dandruff is linked to excess Malassezia yeasts and an imbalance between bacterial and fungal communities on the scalp. Yeast leads to the production of defense substances (cytokines) by the keratinocytes, with the species M. restricta being particularly virulent. Sebum, a choice nutrient for the scalp's inhabitants, produces oxidation products. These damaging substances may promote acceleration of cellular renewal leading to the formation of dandruff. Disorganized and depleted, the stratum corneum no longer plays its protective role.

While consumers seek hair care compositions such as shampoos to care for their hair and treat various scalp conditions such as dandruff, conditioner compositions are typically not recommended for application to the scalp because the components that provide conditioning benefits to the hair fibers may have unwanted effects on the scalp, such as clogging the hair follicles, irritating the scalp, causing buildup of conditioning components on the scalp, and/or causing the hair to have a weighed-down, greasy appearance. Further, to date it has been difficult to formulate a hair conditioner composition that contains scalp care agents such as anti-dandruff active agents that is stable and that effectively provides scalp care as well as benefits such as ease of detangling, smoothness, shine, manageability, and frizz control to the hair.

It has now been found, however, that by using a unique combination of components, compositions that are stable and provide excellent scalp care, while also providing excellent cosmetic properties to hair, can be achieved.

### SUMMARY

The disclosure relates to compositions for caring for keratin materials, and to methods for using the compositions. In various embodiments the compositions are hair conditioner compositions that have scalp care benefits, for example anti-dandruff and/or hydration benefits, as well as hair conditioning benefits.

Compositions according to the disclosure include (a) at least one cationic surfactant, (b) at least one benefit agent, (c) optionally at least one anti-dandruff active agent, and (d) water. The compositions optionally comprise additional components such as fatty acids, moisturizing agents, hair conditioning agents, thickening agents, and auxiliary components. The compositions may, in various embodiments, be compositions for caring for keratin materials such as the hair and/or scalp, or hair conditioner compositions.

In various exemplary embodiments, the compositions are hair conditioner compositions which comprise (a) from about 0.1% to about 15%, such as from about 0.1% to about 10%, from about 0.25% to about 9%, from about 0.5% to about 8%, from about 0.75% to about 7%, or from about 1% to about 6% of cationic surfactants, (b) from about 0.0001% to about 3%, such as from about 0.0001% to about 2.5%, from about 0.0005% to about 2.5%, from about 0.0005% to about 2%, or from about 0.001% to about 1.5% of benefit agents, (c) optionally at least one anti-dandruff active agent, and (d) water. The benefit agents may, for example, be chosen from vitamins, ceramides, sterols, phytosphingosine, and combinations of two or more thereof. In preferred embodiments, the benefit agent comprises ceramide AP, ceramide NP, ceramide EOP, cholesterol, and/or phytosphingosine. If present, the total amount of anti-dandruff active agents ranges from about 0.01% to about 10%, such as from about 0.01% to about 5%, from about 0.05% to about 4%, from about 0.1% to about 3%, or from about 0.1% to about 2% by weight, relative to the total weight of the composition. Useful anti-dandruff active agents include but are not limited to ellagic acid, ethers of ellagic acid, salts of ellagic acid, salts of ethers of ellagic acid, pyrithione, salts of pyrithione, selenium (poly)sulfides, salicylic acid, piroctone olamine, tea tree oil, climbazole, fluocinolone, ketoconazole, coal tar, and combinations thereof. In preferred embodiments, the composition comprises at least one additional component chosen from fatty acids, moisturizing agents, hair conditioning agents, thickening agents, or combinations thereof. Useful fatty acids include, for example, saturated or unsaturated fatty acids having from 8 to 30 carbon atoms, such as unsaturated ω-3 fatty acids having from 16 to 24 carbon atoms and unsaturated ω-6 fatty acids having from 16 to 24 carbon atoms. Non-limiting examples of fatty acids include oleic acid, lauric acid, palmitic acid, myristic acid, stearic acid, linoleic acid, capric acid, caprylic acid, α-linolenic acid, arachidonic acid, eicosapentaenoic acid, docosahexaenoic acid, and combinations thereof. Useful moisturizing agents include, for example, glycols, sugars, glycerol, urea, aloe vera, hyaluronic acid and/or salts thereof, α-hydroxy acids, polyethylene glycols, and combinations thereof. Useful hair conditioning agents include, for example, cationic polymers, silicone compounds, and non-silicone fatty compounds such as plant oils and fatty alcohols.

In further exemplary embodiments, the compositions are for caring for keratin materials such as the hair and/or scalp, and comprise (a) at least one cationic surfactant, (b) at least one benefit agent, (c) at least one anti-dandruff active agent, (d) water, and (e) at least one additional component chosen from fatty acids, moisturizing agents, hair conditioning agents, thickening agents, and combinations thereof. The compositions may, in various embodiments, comprise a total amount of cationic surfactants ranging from about 0.1% to about 15%, such as from about 0.1% to about 10%, from about 0.25% to about 9%, from about 0.5% to about 8%, from about 0.75% to about 7%, or from about 1% to about 6%. The compositions may, in various embodiments, comprise a total amount of benefit agents ranging from about 0.0001% to about 3%, such as from about 0.0001% to about 2.5%, from about 0.0005% to about 2.5%, from about 0.0005% to about 2%, or from about 0.001% to about 1.5% by weight, relative to the total weight of the composition. The benefit agents may, for example, be chosen from vitamins, ceramides, sterols, phytosphingosine, and combinations of two or more thereof. In preferred embodiments, the benefit agent comprises ceramide AP, ceramide NP, ceramide EOP, cholesterol, and/or phytosphingosine. The total amount of anti-dandruff active agents can range from about 0.01% to about 10%, such as from about 0.01% to about 5%, from about 0.05% to about 4%, from about 0.1% to about 3%, or from about 0.1% to about 2% by weight, relative to the total weight of the composition. Useful anti-dandruff active agents include but are not limited to ellagic acid, ethers of ellagic acid, salts of ellagic acid, salts of ethers of ellagic acid, pyrithione, salts of pyrithione, selenium (poly)sulfides, salicylic acid, piroctone olamine, tea tree oil, climbazole, fluocinolone, ketoconazole, coal tar, and combinations thereof. Useful fatty acids include, for example, saturated or unsaturated fatty acids having from 8 to 30 carbon atoms, such as unsaturated ω-3 fatty acids having from 16 to 24 carbon atoms and unsaturated ω-6 fatty acids having from 16 to 24 carbon atoms. Non-limiting examples of fatty acids include oleic acid, lauric acid, palmitic acid, myristic acid, stearic acid, linoleic acid, capric acid, caprylic acid, α-linolenic acid, arachidonic acid, eicosapentaenoic acid, docosahexaenoic acid, and combinations thereof. Useful moisturizing agents include, for example, glycols, sugars, glycerol, urea, aloe vera, hyaluronic acid and/or salts thereof, α-hydroxy acids, polyethylene glycols, and combinations thereof. Useful hair conditioning agents include, for example, cationic polymers, silicone compounds, and non-silicone fatty compounds such as plant oils and fatty alcohols.

The disclosure further relates to methods of using the compositions for caring for keratin materials. The methods comprise applying the compositions to keratin materials such as the hair and/or scalp, and optionally rinsing the composition from the keratin materials. In various embodiments, the compositions may be left on the hair for an optional leave-in time, for example a period of time sufficient to allow the components in the composition to provide various benefits to the hair and/or scalp. In some embodiments the compositions are applied to the keratin materials as part of a personal care routine, where the hair and/or scalp may be cleansed with a shampoo composition prior to treatment of the hair and/or scalp with a composition according to the disclosure.

### BRIEF DESCRIPTION OF THE FIGURES

FIG. 1 shows a comparison of various properties of compositions according to the disclosure and commercially-available compositions, and hair treated with the compositions in a cleansing and conditioning routine.

FIG. 2 is an image of hair treated with a composition according to the disclosure (side A) compared to a commercially-available composition (side B) during a cleansing and conditioning routine.

FIG. 3 is an image of hair treated with a composition according to the disclosure (side A) compared to a commercially-available composition (side B) after a cleansing and conditioning routine.

FIG. 4 is an image of hair treated with a composition according to the disclosure (side A) compared to a commercially-available composition (side B) after a cleansing and conditioning routine.

### DETAILED DESCRIPTION

The disclosure relates to compositions for caring for the keratin materials such as the hair and scalp, and to methods of caring for keratin materials with the compositions.

### I. COMPOSITIONS

Compositions according to the disclosure include (a) at least one cationic surfactant, (b) at least one benefit agent, (c) optionally at least one anti-dandruff active agent, and (d) at least one solvent. The compositions may optionally comprise additional components such as fatty acids, moisturizing agents, conditioning agents, thickening agents, and auxiliary components.

### Cationic Surfactants

Compositions according to the disclosure comprise at least one cationic surfactant. Salts of cationic surfactants are expressly included, whether or not stated. Exemplary and non-limiting cationic surfactants include cationic amine-based or quaternary ammonium-based compounds.

For example, cationic surfactants may be chosen from alkylpyridinium salts, ammonium salts of imidazoline, diquaternary ammonium salts, and ammonium salts containing at least one ester function. As a further example, cationic surfactants may be chosen from quaternary ammonium salts having the following formula (I):

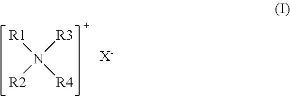

wherein R1 to R4, which may be identical or different, represent a linear or branched aliphatic radical containing from 1 to 30 carbon atoms, or an aromatic radical such as aryl or alkylaryl; the aliphatic radicals may optionally comprise heteroatoms (O, N, S or halogens) and may optionally be substituted, and X⁻ is an anion chosen from the group of halides, phosphates, acetates, lactates, C2-C6 alkyl sulfates and alkyl or alkylarylsulfonates. The aliphatic radicals are chosen, for example, from C12-C22 alkyl, alkoxy, C2-C6 polyoxyalkylene, alkylamide, (C12-C22) alkylamido (C2-C6) alkyl, (C12-C22) alkyl-acetate and hydroxyalkyl radicals, containing from 1 to 30 carbon atoms.

As a further example, quaternary ammonium salts containing at least one ester function, such as those of formula (II) may be chosen:

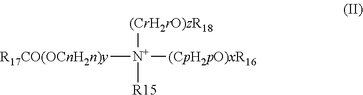

wherein:
   R15 is chosen from C1-C6 alkyl radicals and C1-C6 hydroxyalkyl or dihydroxyalkyl radicals;
   R16 is chosen from the radical R19-CO—, linear or branched, saturated or unsaturated C1-C22 hydrocarbon-based radicals R20, a hydrogen atom;
   R18 is chosen from the radical R21-CO, linear or branched, saturated or unsaturated C1-C22 hydrocarbon-based radicals R22, a hydrogen atom;
   R17, R19 and R21, which may be identical or different, are chosen from linear or branched, saturated or unsaturated C7-C21 hydrocarbon-based radicals;
   r, n, and p, which may be identical or different, are integers ranging from 2 to 6;
   y is an integer ranging from 1 to 10;
   x and z, which may be identical or different, are integers ranging from 0 to 10; and
   X⁻ is a simple or complex organic or mineral anion;
with the provisos that:
   the sum x+y+z is from 1 to 15,
   when x is 0, then R16 denotes R20, and
   when z is 0, then R18 denotes R22.

In formula (II), the alkyl radical R15 may be linear or branched, and more particularly linear. Preferably, R15 denotes a methyl, ethyl, hydroxyethyl or dihydroxypropyl radical, and more particularly a methyl or ethyl radical. Advantageously, the sum x+y+z is from 1 to 10. When R16 is a hydrocarbon-based radical R20, it may contain from 12 to 22 carbon atoms, or contain from 1 to 3 carbon atoms. When R18 is a hydrocarbon-based radical R22, it preferably contains 1 to 3 carbon atoms. Advantageously, R17, R19, and R21, which may be identical or different, are chosen from linear or branched, saturated or unsaturated C11-C21 hydrocarbon-based radicals, and more particularly from linear or branched, saturated or unsaturated C11-C21 alkyl and alkenyl radicals. Preferably, x and z, which may be identical or different, are equal to 0 or 1. Advantageously, y is equal to 1. Preferably, r, n, and p, which may be identical or different, are equal to 2 or 3 and even more particularly equal to 2. The anion X⁻ is preferably a halide (chloride, bromide or iodide) or a C1-C4 alkyl sulfate, more particularly methyl sulfate. The anion X may also represent methanesulfonate, phosphate, nitrate, tosylate, an anion derived from an organic acid (such as acetate or lactate), or any other anion that is compatible with the ammonium containing an ester function.

The surfactants may be, for example, the salts (chloride or methyl sulfate) of diacyloxyethyldimethylammonium, of diacyloxyethylhydroxyethyldimethylammonium, of monoacyloxyethylhydroxyethyldimethylammonium, of triacyloxyethylmethylammonium, of monoacyloxyethylhydroxyethyldimethylammonium, and mixtures thereof. The acyl radicals preferably contain 14 to 18 carbon atoms and are more particularly derived from a plant oil, for instance palm oil or sunflower oil. When the compound contains several acyl radicals, these radicals may be identical or different.

Other suitable cationic surfactants are esterquats which are quaternary ammonium compounds having fatty acid chains containing ester linkages, such as, for example, dibehenoylethyl dimonium chloride, dipalmitoylethyl dimonium chloride, distearoylethyl dimonium chloride, ditallowoyl PG-dimonium chloride, dipalmitoylethyl hydroxyethylmonium methosulfate, distearoylethyl hydroxyethylmonium methosulfate, or mixtures thereof.

In preferred embodiments, the cationic surfactant is chosen from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyl-diethylamine, behenamidoethyldiethylamine, behenamidoethyldimethylamine, arachidamidopropyldimethylamine, arachidamidopropyidiethylamine, arachidamidoethyl-diethylamine, arachidamidoethyidimethylamine, and combinations of two or more thereof. For example, the cationic surfactants may comprise, consist essentially of, or consist of cetrimonium chloride, behentrimonium chloride, and/or behentrimonium methosulfate.

The total amount of cationic surfactants present in the composition may vary, but typically ranges from about 0.1% to about 15%, such as from about 0.1% to about 10%, from about 0.25% to about 9%, from about 0.5% to about 8%, from about 0.75% to about 7%, or from about 1% to about 6% by weight, relative to the total weight of the composition. For example, the composition may comprise a total amount of cationic surfactants ranging from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.75% to about 10%, from about 0.75% to about 9%, from about 0.75% to about 8%, from about 0.75% to about 7%, from about 0.75% to about 6%, from about 0.75% to about 5%, from about 0.75% to about 4%, from about 1% to about 10%, from about 1% to about 9%, from about 1% to about 8%, from about 1% to about 7%, from about 1% to about 6%, from about 1% to about 5%, from about 1% to about 4%, from about 1.25% to about 10%, from about 1.25% to about 9%, from about 1.25% to about 8%, from about 1.25% to about 7%, from about 1.25% to about 6%, from about 1.25% to about 5%, from about 1.25% to about 4%, from about 1.5% to about 10%, from about 1.5% to about 9%, from about 1.5% to about 8%, from about 1.5% to about 7%, from about 1.5% to about 6%, from about 1.5% to about 5%, from about 1.5% to about 4%, from about 1.75% to about 10%, from about 1.75% to about 9%, from about 1.75% to about 8%, from about 1.75% to about 7%, from about 1.75% to about 6%, from about 1.75% to about 5%, from about 1.75% to about 4%, from about 2% to about 10%, from about 2% to about 9%, from about 2% to about 8%, from about 2% to about 7%, from about 2% to about 6%, from about 2% to about 5%, from about 2% to about 4%, from about 2.5% to about 9%, from about 2.5% to about 8%, from about 2.5% to about 7%, from about 2.5% to about 6%, from about 2.5% to about 5%, from about 2.5% to about 4%, from about 3% to about 10%, from about 3% to about 9%, from about 3% to about 8%, from about 3% to about 7%, from about 3% to about 6%, from about 3% to about 5%, from about 3% to about 4%, from about 3.5% to about 9%, from about 3.5% to about 8%, from about 3.5% to about 7%, from about 3.5% to about 6%, from about 3.5% to about 5%, or from about 3.5% to about 4% by weight, relative to the total weight of the composition. For example, in some embodiments, the compositions comprise about 1%, about 1.5%, about 2%, about 2.5%, about 3%, about 3.5%, about 4%, about 4.5%, about 5%, about 5.5%, about 6%, about 6.5%, about 7%, about 7.5%, or about 8% of cationic surfactants, or may comprise a total amount of cationic surfactants using any of the foregoing as upper or lower limits.

Although the compositions may comprise at least one surfactant other than cationic surfactants, preferably the total amount of such surfactants individually or in combination is less than about 5% by weight, relative to the total weight of the composition. For example, the compositions may comprise less than about 5%, less than about 4%, less than about 3%, less than about 2%, less than about 1%, or less than about 0.5% of the total of all surfactants other than cationic surfactants; less than about 5%, less than about 4%, less than about 3%, less than about 2%, less than about 1%, or less than about 0.5% of the total of all anionic surfactants; less than about 5%, less than about 4%, less than about 3%, less than about 2%, less than about 1%, or less than about 0.5% of the total of all amphoteric surfactants; and/or less than about 5%, less than about 4%, less than about 3%, less than about 2%, less than about 1%, or less than about 0.5% of the total of all nonionic surfactants.

The compositions according to the disclosure are not cleansing compositions. Therefore, in particular embodiments, the total amount of surfactants that those of skill in the art would consider to be cleansing surfactants is less than about 5%, such as less than about 4%, less than about 3%, less than about 2%, less than about 1%, or less than about 0.5% by weight, relative to the total weight of the composition.

### Benefit Agents

Compositions according to the disclosure comprise at least one benefit agent. Benefit agents provide benefits to the scalp such as improved skin barrier functionality, or promote regeneration, renewal, repair and/or improved moisture retention of the scalp, and also provide benefits to the hair such as improved smoothness and frizz control. Non-limiting examples of benefit agents that may be chosen include sterols such as cholesterol and lanosterol, vitamins and analogues thereof such as B1 (thiamine), B3 (niacinamide), B5 (pantothenic acid), and E (tocopherol acetate), phytosphingosine, ceramides such as ceramide AP, ceramide NP, ceramide EOP, and combinations of two or more thereof.

While the total amount of benefit agents may vary, compositions will typically include from about 0.0001% to about 3% by weight, relative to the total weight of the composition. For example, in various embodiments, the total amount of benefit agents ranges from about 0.0001% to about 2.5%, such as from about 0.0001% to about 2%, from about 0.0001% to about 1.5%, from about 0.0001% to about 1.25%, from about 0.0001% to about 1%, from about 0.0001% to about 0.75%, from about 0.0001% to about 0.5%, from about 0.0001% to about 0.4%, from about 0.0001% to about 0.3%, from about 0.0001% to about 0.2%, from about 0.0001% to about 0.1%, from about 0.0001% to about 0.05%, from about 0.0005% to about 3%, from about 0.0005% to about 2.5%, from about 0.0005% to about 2%, from about 0.0005% to about 1.5%, from about 0.0005% to about 1.25%, from about 0.0005% to about 1%, from about 0.0005% to about 0.75%, from about 0.0005% to about 0.5%, from about 0.0005% to about 0.4%, from about 0.0005% to about 0.3%, from about 0.0005% to about 0.2%, from about 0.0005% to about 0.1%, from about 0.0005% to about 0.05%, from about 0.001% to about 3%, from about 0.001% to about 2.5%, from about 0.001% to about 2%, from about 0.001% to about 1.5%, from about 0.001% to about 1.25%, from about 0.001% to about 1%, from about 0.001% to about 0.75%, from about 0.001% to about 0.5%, from about 0.001% to about 0.4%, from about 0.001% to about 0.3%, from about 0.001% to about 0.2%, from about 0.001% to about 0.1%, from about 0.001% to about 0.05%, from about 0.005% to about 3%, from about 0.005% to about 2.5%, from about 0.005% to about 2%, from about 0.005% to about 1.5%, from about 0.005% to about 1.25%, from about 0.005% to about 1%, from about 0.005% to about 0.75%, from about 0.005% to about 0.5%, from about 0.005% to about 0.4%, from about 0.005% to about 0.3%, from about 0.005% to about 0.2%, from about 0.005% to about 0.1%, from about 0.005% to about 0.05%, from about 0.01% to about 1%, from about 0.01% to about 0.75%, from about 0.01% to about 0.5%, from about 0.01% to about 0.4%, from about 0.01% to about 0.3%, from about 0.01% to about 0.2%, from about 0.01% to about 0.1%, from about 0.01% to about 0.05%, from about 0.05% to about 3%, from about 0.05% to about 2.5%, from about 0.05% to about 2%, from about 0.05% to about 1.5%, from about 0.05% to about 1.25%, from about 0.05% to about 1%, from about 0.05% to about 0.75%, from about 0.05% to about 0.5%, from about 0.05% to about 0.4%, from about 0.05% to about 0.3%, from about 0.05% to about 0.2%, from about 0.05% to about 0.1%, from about 0.1% to about 1%, from about 0.1% to about 0.75%, from about 0.1% to about 0.5%, from about 0.1% to about 0.4%, from about 0.1% to about 0.3%, from about 0.1% to about 0.2%, from about 0.5% to about 3%, from about 0.5% to about 2.5%, from about 0.5% to about 2%, from about 0.5% to about 1.5%, from about 0.5% to about 1.25%, from about 0.5% to about 1%, or from about 0.5% to about 0.75% by weight, relative to the total weight of the composition. In certain embodiments, the compositions comprise a total amount of benefit agents in any of the aforementioned ranges, wherein the benefit agent comprises one or more of ceramide AP, ceramide NP, and ceramide EOP.

### Anti-Dandruff Active Agents

The compositions optionally comprise at least one anti-dandruff active agent. In combination with the benefit agents, the anti-dandruff active agents can, in various embodiments, provide enhanced scalp care. Non-limiting examples of useful anti-dandruff active agents include ellagic acid, ethers of ellagic acid, salts of ellagic acid, salts of ethers of ellagic acid, salts of pyrithione, 1-hydroxy-2-pyridone derivatives, selenium (poly)sulfides, salicylic acid, piroctone olamine, tea tree oil, climbazole, fluocinolone, ketoconazole, coal tar, or combinations of two or more thereof.

By way of example, useful ellagic acid ethers can be chosen from the mono-, di-, tri- or polyethers obtained by etherification of one or more —OH groups of ellagic acid to give one or more —OR groups, where R is chosen from C2-C20 alkyl groups, polyoxyalkylene groups such as polyoxyethylene and/or polyoxypropylene groups, and groups derived from one or more mono- or polysaccharides. In the case of the di-, tri- or polyethers of ellagic acid, the R groups as defined above can be identical or different. For example, ethers of ellagic acid such as 3,4-di-O-methyl ellagic acid, 3,3′,4-tri-O-methyl ellagic acid, or 3,3′-di-O-methyl ellagic acid may be chosen.

Useful and non-limiting salts of ellagic acid and/or of its ethers can be chosen from alkali metal or alkaline earth metal salts, such as the sodium, potassium, calcium, or magnesium salts, the ammonium salt, and the salts of amines, such as triethanolamine, monoethanolamine, arginine, and lysine salts.

In various embodiments, pyrithione salts are chosen from monovalent metal salts or divalent metal salts, such as the sodium, calcium, magnesium, barium, strontium, zinc, cadmium, tin, or zirconium salts. In some embodiments, the zinc salt (zinc pyrithione) is particularly useful.

As non-limiting examples of 1-hydroxy-2-pyridone derivatives, compounds of formula (Z) or salts thereof may be chosen:

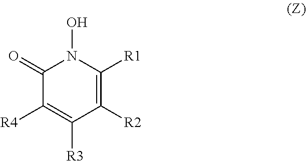

wherein:
   R1 is chosen from a hydrogen atom; a linear or branched alkyl group having from 1 to 17 carbon atoms; a cycloalkyl group having from 5 to 8 carbon atoms; a cycloalkyl-alkyl group, the cycloalkyl group having from 5 to 8 carbon atoms and the alkyl group having from 1 to 4 carbon atoms; an aryl or aralkyl group, the aryl group having from 6 to 30 carbon atoms and the alkyl group having from 1 to 4 carbon atoms; an aryl-alkenyl group, the aryl group having from 6 to 30 carbon atoms and the alkenyl group having from 2 to 4 carbon atoms; it being possible for the cycloalkyl and aryl groups as defined above to be substituted by one or more alkyl groups having from 1 to 4 carbon atoms or else one or more alkoxy groups having from 1 to 4 carbon atoms;
   R2 is chosen from a hydrogen atom; an alkyl group having from 1 to 4 carbon atoms; an alkenyl group having from 2 to 4 carbon atoms; a halogen atom or a benzyl group;
   R3 is chosen from a hydrogen atom; an alkyl group having from 1 to 4 carbon atoms or a phenyl group; and
   R4 is chosen from a hydrogen atom; an alkyl group having from 1 to 4 carbon atoms; an alkenyl group having from 2 to 4 carbon atoms; a methoxymethyl group; a halogen atom or a benzyl group.

By way of example, 1-hydroxy-4-methyl-6-(2,4,4-trimethylpentyl)-2(1H)-pyridone or 6-cyclohexyl-1-hydroxy-4-methyl-2(1H)-pyridone may be chosen.

Useful salts include salts of C1-C4 alkanolamines, such as monoethanolamine or diethanolamine, amine or alkylamine salts, and also salts with inorganic cations, such as ammonium salts and the salts of alkali metals or alkaline earth metals. For example, in some embodiments, the monoethanolamine salt of 1-hydroxy-4-methyl-6-(2,4,4-trimethylpentyl)-2(1H)-pyridinone (or piroctone), commonly referred to as piroctone olamine, can be chosen.

Non-limiting examples of selenium (poly)sulfides include selenium disulfide and the selenium polysulfides having the formula SeₓS_{y}, in which x and y are numbers such that x+y=8.

If present, the total amount of anti-dandruff active agents may vary, but typically ranges from about 0.01% to about 10%, including all ranges and subranges therebetween, such as from about 0.01% to about 5%, from about 0.01% to about 4%, from about 0.01% to about 3%, from about 0.01% to about 2%, from about 0.01% to about 1.5%, from about 0.01% to about 1.25%, from about 0.01% to about 1%, from about 0.01% to about 0.75%, from about 0.1% to about 5%, from about 0.1% to about 4%, from about 0.1% to about 3%, from about 0.1% to about 2%, from about 0.1% to about 1.5%, from about 0.1% to about 1.25%, from about 0.1% to about 1%, from about 0.1% to about 0.75%, from about 0.5% to about 5%, from about 0.5% to about 4%, from about 0.5% to about 3%, from about 0.5% to about 2%, from about 0.5% to about 1.5%, from about 0.5% to about 1.25%, from about 0.5% to about 1%, or from about 0.5% to about 0.75% by weight, relative to the total weight of the composition.

### Fatty Acids

Compositions according to the disclosure optionally comprise at least one fatty acid. Without intending to be limiting, it is believed that fatty acids also enhance the hair and scalp care benefits of compositions according to the disclosure. In some embodiments, the compositions comprise at least two fatty acids, at least three fatty acids, or more.

Fatty acids comprise an aliphatic hydrocarbon chain with a carboxyl group (—COOH) at one end, and a methyl group (—CH3) at the other end. Fatty acids that can be chosen include saturated or unsaturated, linear or branched fatty acids. For example, in some embodiments the fatty acids are chosen from those having from 6 to 30 carbon atoms, such as from 8 to 26, from 10 to 24, from 10 to 22, from 10 to 20, from 12 to 30, from 12 to 24, from 12 to 22, from 12 to 20, from 14 to 30, from 14 to 24, from 14 to 22, from 14 to 20, from 15 to 30, from 15 to 24, from 15 to 22, from 15 to 20, from 16 to 30, from 16 to 24, from 16 to 22, or from 16 to 20 carbon atoms, and which are saturated or unsaturated. When the fatty acids are unsaturated, they may be in cis- or trans- configuration.

In at least some embodiments, the fatty acid(s) comprise, consist essentially of, or consist of saturated or unsaturated, linear or branched fatty acids with at least 12 or at least 15 carbon atoms, for example having from 16 to 30 carbon atoms, from 16 to 24 carbon atoms, from 16 to 22 carbon atoms, or from 16 to 20 carbon atoms. In some embodiments, the fatty acid(s) comprise, consist essentially of, or consist of saturated or unsaturated, linear fatty acids with at least 12 or at least 15 carbon atoms, for example having from 16 to 30 carbon atoms, from 16 to 24 carbon atoms, from 16 to 22 carbon atoms, or from 16 to 20 carbon atoms.

In some embodiments, the fatty acid comprises at least one monounsaturated and/or at least one polyunsaturated fatty acid, which may be in cis- or trans- configuration. In some embodiments, the fatty acid comprises at least one polyunsaturated fatty acid. For example, in some embodiments the composition comprises at least one monounsaturated and/or at least one polyunsaturated fatty acid which have a double bond between the third and fourth carbon from the methyl end of the chain (ω-3 fatty acids), and/or between the sixth and seventh carbon from the methyl end of the chain (ω-6 fatty acids), and/or between the ninth and tenth carbon from the methyl end of the chain (ω-9 fatty acids). In some embodiments, the composition comprises at least one unsaturated ω-3 fatty acid and/or at least one unsaturated ω-6 fatty acid. In further embodiments, the composition comprises at least one unsaturated ω-3 fatty acid having from 16 to 30 carbon atoms, such as from 16 to 24 carbon atoms, from 16 to 22 carbon atoms, or from 16 to 20 carbon atoms, and/or at least one unsaturated ω-6 fatty acid having from 16 to 30 carbon atoms, such as from 16 to 24 carbon atoms, from 16 to 22 carbon atoms, or from 16 to 20 carbon atoms.

Non-limiting examples of fatty acids that can be chosen include oleic acid, lauric acid, palmitic acid, myristic acid, stearic acid, linoleic acid, capric acid, caprylic acid, α-linolenic acid, γ-linolenic acid, pentadecanoic acid, palmitoleic acid, arachidonic acid, eicosapentaenoic acid, docosahexaenoic acid, erucic acid, or combinations of two or more thereof. In some embodiments, the fatty acids comprise at least one fatty acid chosen from capric acid, caprylic acid, oleic acid, myristic acid, palmitic acid, stearic acid, linoleic acid, and α-linolenic acid. In some embodiments, the fatty acid comprises, consists essentially of, or consists of oleic acid, palmitic acid, stearic acid, linoleic acid, α-linolenic acid, or a combination of two or more thereof, for example linoleic acid and α-linolenic acid. In some embodiments, the fatty acid comprises, consists essentially of, or consists of one or more polyunsaturated fatty acids. In some embodiments, the fatty acid comprises at least one monounsaturated fatty acid and at least one polyunsaturated fatty acid, and optionally at least one saturated fatty acid.

The total amount of fatty acids may vary, but typically ranges from about 0.001% to about 3%, such as from about 0.01% to about 2%, from about 0.05% to about 1.5%, from about 0.1% to about 1.25%, or from about 0.1% to about 1% by weight, based on the total weight of the composition. In various embodiments, the total amount of fatty acids may range from about 0.01% to about 3%, from about 0.01% to about 2.75%, from about 0.01% to about 2.5%, from about 0.01% to about 2.25%, from about 0.01% to about 2%, from about 0.01% to about 1.75%, from about 0.01% to about 1.5%, from about 0.01% to about 1.25%, from about 0.01% to about 1.2%, from about 0.01% to about 1.1%, from about 0.01% to about 1.0%, from about 0.01% to about 0.9%, from about 0.01% to about 0.8%, from about 0.01% to about 0.7%, from about 0.01% to about 0.6%, from about 0.01% to about 0.5%, from about 0.01% to about 0.4%, from about 0.01% to about 0.3%, from about 0.01% to about 0.2%, from about 0.01% to about 0.1%, from about 0.05% to about 3%, from about 0.05% to about 2.75%, from about 0.05% to about 2.5%, from about 0.05% to about 2.25%, from about 0.05% to about 2%, from about 0.05% to about 1.75%, from about 0.05% to about 1.5%, from about 0.05% to about 1.25%, from about 0.05% to about 1.2%, from about 0.05% to about 1.1%, from about 0.05% to about 1.0%, from about 0.05% to about 0.9%, from about 0.05% to about 0.8%, from about 0.05% to about 0.7%, from about 0.05% to about 0.6%, from about 0.05% to about 0.5%, from about 0.05% to about 0.4%, from about 0.05% to about 0.3%, from about 0.05% to about 0.2%, from about 0.05% to about 0.1%, from about 0.1% to about 3%, from about 0.1% to about 2.75%, from about 0.1% to about 2.5%, from about 0.1% to about 2.25%, from about 0.1% to about 2%, from about 0.1% to about 1.75%, from about 0.1% to about 1.5%, from about 0.1% to about 1.25%, from about 0.1% to about 1.2%, from about 0.1% to about 1.1%, from about 0.1% to about 1.0%, from about 0.1% to about 0.9%, from about 0.1% to about 0.8%, from about 0.1% to about 0.7%, from about 0.1% to about 0.6%, from about 0.1% to about 0.5%, from about 0.1% to about 0.4%, from about 0.1% to about 0.3%, from about 0.1% to about 0.2%, from about 0.15% to about 3%, from about 0.15% to about 2.75%, from about 0.15% to about 2.5%, from about 0.15% to about 2.25%, from about 0.15% to about 2%, from about 0.15% to about 1.75%, from about 0.15% to about 1.5%, from about 0.15% to about 1.25%, from about 0.15% to about 1.2%, from about 0.15% to about 1.1%, from about 0.15% to about 1.0%, from about 0.15% to about 0.9%, from about 0.15% to about 0.8%, from about 0.15% to about 0.7%, from about 0.15% to about 0.6%, from about 0.15% to about 0.5%, from about 0.15% to about 0.4%, from about 0.15% to about 0.3%, from about 0.15% to about 0.2%, from about 0.2% to about 3%, from about 0.2% to about 2.75%, from about 0.2% to about 2.5%, from about 0.2% to about 2.25%, from about 0.2% to about 2%, from about 0.2% to about 1.75%, from about 0.2% to about 1.5%, from about 0.2% to about 1.25%, from about 0.2% to about 1.2%, from about 0.2% to about 1.1%, from about 0.2% to about 1.0%, from about 0.2% to about 0.9%, from about 0.2% to about 0.8%, from about 0.2% to about 0.7%, from about 0.2% to about 0.6%, from about 0.2% to about 0.5%, from about 0.2% to about 0.4%, from about 0.2% to about 0.3%, from about 0.25% to about 3%, from about 0.25% to about 2.75%, from about 0.25% to about 2.5%, from about 0.25% to about 2.25%, from about 0.25% to about 2%, from about 0.25% to about 1.75%, from about 0.25% to about 1.5%, from about 0.25% to about 1.25%, from about 0.25% to about 1.2%, from about 0.25% to about 1.1%, from about 0.25% to about 1.0%, from about 0.25% to about 0.9%, from about 0.25% to about 0.8%, from about 0.25% to about 0.7%, from about 0.25% to about 0.6%, from about 0.25% to about 0.5%, from about 0.25% to about 0.4%, from about 0.25% to about 0.3%, from about 0.3% to about 3%, from about 0.3% to about 2.75%, from about 0.3% to about 2.5%, from about 0.3% to about 2.25%, from about 0.3% to about 2%, from about 0.3% to about 1.75%, from about 0.3% to about 1.5%, from about 0.3% to about 1.25%, from about 0.3% to about 1.2%, from about 0.3% to about 1.1%, from about 0.3% to about 1.0%, from about 0.3% to about 0.9%, from about 0.3% to about 0.8%, from about 0.3% to about 0.7%, from about 0.3% to about 0.6%, from about 0.3% to about 0.5%, from about 0.3% to about 0.4% by weight, based on the total weight of the composition. In further embodiments, the compositions comprise one or more fatty acids in a total amount of about 0.07%, about 0.08%, about 0.09%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 1.1%, about 1.2%, about 1.3%, about 1.4%, about 1.5%, about 1.55%, about 1.6%, about 1.65%, or about 1.7% by weight, based on the total weight of the composition, or in a range using any of the foregoing as upper and lower limits.

In some embodiments, the compositions comprise a total amount of fatty acids in any of the aforementioned amounts or ranges, and include at least one unsaturated fatty acid, for example at least one polyunsaturated fatty acid such as linoleic acid and/or α-linolenic acid. In some embodiments, the compositions comprise a total amount of unsaturated fatty acids in any of the aforementioned amounts or ranges.

### Moisturizing Agents

In some embodiments, the compositions comprise one or more agents that can provide moisturizing benefits to the hair and/or scalp. Advantageously, in some embodiments the compositions comprise moisturizing agents with hydrophilic residues. Without intending to be limiting, it is believed that hydrophilic moisturizing agents enhance the hair and scalp care benefits of compositions according to the disclosure.

Non-limiting examples of useful moisturizers include glycols such as propylene glycol, hexylene glycol, caprylyl glycol, and butylene glycol, glyceryl triacetate, glycerol (glycerin), sugars such as sorbitol, xylitol, maltitol, and polydextrose, hydrogenated starch hydrolysate, urea, aloe vera, hyaluronic acid and/or salts thereof such as sodium hyaluronate, α-hydroxy acids, polyethylene glycols such as PEG-4, PEG-6, PEG-7, PEG-8, PEG-9, PEG-10, PEG-12, PEG-14, PEG-16, PEG-18, PEG-20, PEG-32, PEG-40, PEG-55, PEG-60, PEG-75, PEG-90, PEG-100, PEG-135, PEG-150, PEG-154, PEG-180, PEG-200, and PEG-240, or combinations of two or more thereof.

If present, the total amount of moisturizing agents in the composition may vary, but typically ranges from about 0.1% to about 10%, such as from about 0.25% to about 9%, from about 0.5% to about 8%, from about 0.75% to about 7%, or from about 1% to about 6% by weight, relative to the total weight of the composition. For example, the composition may comprise a total amount of moisturizing agents ranging from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.75% to about 10%, from about 0.75% to about 9%, from about 0.75% to about 8%, from about 0.75% to about 7%, from about 0.75% to about 6%, from about 0.75% to about 5%, from about 0.75% to about 4%, from about 1% to about 10%, from about 1% to about 9%, from about 1% to about 8%, from about 1% to about 7%, from about 1% to about 6%, from about 1% to about 5%, from about 1% to about 4%, from about 1.25% to about 10%, from about 1.25% to about 9%, from about 1.25% to about 8%, from about 1.25% to about 7%, from about 1.25% to about 6%, from about 1.25% to about 5%, from about 1.25% to about 4%, from about 1.5% to about 10%, from about 1.5% to about 9%, from about 1.5% to about 8%, from about 1.5% to about 7%, from about 1.5% to about 6%, from about 1.5% to about 5%, from about 1.5% to about 4%, from about 1.75% to about 10%, from about 1.75% to about 9%, from about 1.75% to about 8%, from about 1.75% to about 7%, from about 1.75% to about 6%, from about 1.75% to about 5%, from about 1.75% to about 4%, from about 2% to about 10%, from about 2% to about 9%, from about 2% to about 8%, from about 2% to about 7%, from about 2% to about 6%, from about 2% to about 5%, from about 2% to about 4%, from about 2.5% to about 9%, from about 2.5% to about 8%, from about 2.5% to about 7%, from about 2.5% to about 6%, from about 2.5% to about 5%, from about 2.5% to about 4%, from about 3% to about 10%, from about 3% to about 9%, from about 3% to about 8%, from about 3% to about 7%, from about 3% to about 6%, from about 3% to about 5%, from about 3% to about 4%, from about 3.5% to about 9%, from about 3.5% to about 8%, from about 3.5% to about 7%, from about 3.5% to about 6%, from about 3.5% to about 5%, or from about 3.5% to about 4% by weight, relative to the total weight of the composition.

### Hair Conditioning Agents

The compositions optionally comprise one or more hair conditioning agents. If present, hair conditioning agents may be chosen from, for example, cationic conditioning agents, silicone compounds, non-silicone fatty compounds, or combinations thereof.

Cationic conditioning agents include, by way of example, polymers having at least one amine group chosen from primary, secondary, tertiary, and quaternary amine groups, and such groups may either form part of the main polymer chain or may be borne by a side substituent that is directly attached to the main polymer chain. In various embodiments, cationic conditioning agents may be chosen from amidoamines, monoalkyl quaternary amines, dialkyl quaternary amines, polyquaternium compounds, or salts thereof.

Examples of amidoamines include, but are not limited to, oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamindopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicamidopropyl dimethylamine, olivamidopropyl dimethylamine, palmitamidopropyl dimethylamine, stearamidoethyldiethylamine, and mixtures thereof.

Non-limiting examples of polyquaternium compounds include polyquaternium 5, polyquaternium 6, polyquaternium 7, polyquaternium 10, polyquaternium 11, polyquaternium 15, polyquaternium 16, polyquaternium 22, polyquaternium 28, polyquaternium 39, polyquaternium 44, polyquaternium 46, polyquaternium 47, and mixtures thereof.

Useful and non-limiting silicone conditioning agents that can be included in compositions according to the disclosure include, but are not limited to, polyorganosiloxanes, polyalkylsiloxanes, polyarylsiloxanes, polyalkarylsiloxanes, polyestersiloxanes, and mixtures thereof. For example, dimethicone, cyclomethicone (cyclopentasiloxane), amodimethicone, trimethyl silyl amodimethicone, phenyl trimethicone, trimethyl siloxy silicate, polymethylsilsesquioxane, or mixtures thereof may be chosen.

In some embodiments, however, the compositions according to the disclosure are free or essentially free of silicones. For example, the compositions may in some embodiments include less than about 3%, less than about 2%, less than about 1.75%, less than about 1.5%, less than about 1.25%, less than about 1%, less than about 0.75%, less than about 0.5%, less than about 0.25%, less than about 0.1%, less than about 0.05%, or less than about 0.01% of silicones.

Useful and non-limiting examples of non-silicone fatty compounds include, for example, lower alkanes, fatty alcohols, esters of fatty acids, esters of fatty alcohols, or combinations thereof. By way of example, non-silicone fatty compounds may be chosen from plant oils such as sweet almond oil, avocado oil, castor oil, olive oil, jojoba oil, sunflower oil, wheat germ oil, sesame oil, groundnut oil, grape seed oil, soybean oil, rapeseed oil, passion-flower oil, coconut oil, corn oil, hazelnut oil, karite butter, palm oil, and apricot kernel oil; hydrocarbons, such as mineral oil and hydrogenated polyisobutene; fatty alcohols, such as octyldodecanol; esters, such as C12-C15 alkyl benzoate; diesters, such as propylene dipelarganate; and triesters, such as glyceryl trioctanoate. Examples of useful low viscosity oils include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, or combinations of octyldodecanol, acetylated lanolin alcohol, cetyl acetate, isododecanol, polyglyceryl-3-diisostearate, or mixtures thereof. Examples of useful high viscosity oils include castor oil, lanolin and lanolin derivatives, triisocetyl citrate, sorbitan sesquioleate, C10-C18 triglycerides, caprylic/capric/triglycerides, coconut oil, corn oil, cottonseed oil, glyceryl triacetyl hydroxystearate, glyceryl triacetyl ricinoleate, glyceryl trioctanoate, hydrogenated castor oil, linseed oil, mink oil, olive oil, palm oil, illipe butter, rapeseed oil, soybean oil, sunflower seed oil, tallow, tricaprin, trihydroxystearin, triisostearin, trilaurin, trilinolein, trimyristin, triolein, tripalmitin, tristearin, walnut oil, wheat germ oil, stearyl alcohol, cetyl alcohol, cetearyl alcohol, or combinations of two or more thereof. In some embodiments, the compositions comprise at least one non-silicone fatty compound, for example cetearyl alcohol and/or cetyl esters.

In some embodiments, the compositions comprise at least one conditioning agent chosen from silicone compounds and non-silicone fatty compounds. For example, the compositions may comprise at least one silicone compound and/or at least one fatty alcohol.

When present, the total amount of hair conditioning agents can vary, but will typically be present in a total amount ranging from about 0.01% to about 12%, such as from about 0.1% to about 10%, from about 0.5% to about 9%, from about 1% to about 8%, or from about 2% to about 7% by weight, relative to the total weight of the composition. For example, the total amount of hair conditioning agents may range from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 1% to about 9%, from about 1% to about 8%, from about 1% to about 7%, from about 1% to about 6%, from about 1% to about 5%, from about 1% to about 4%, from about 1% to about 3%, from about 1.5% to about 9%, from about 1.5% to about 8%, from about 1.5% to about 7%, from about 1.5% to about 6%, from about 1.5% to about 5%, from about 1.5% to about 4%, from about 1.5% to about 3%, from about 2% to about 9%, from about 2% to about 8%, from about 2% to about 7%, from about 2% to about 6%, from about 2% to about 5%, from about 2% to about 4%, or from about 2% to about 3% by weight, relative to the total weight of the composition.

### Thickening Agents/Rheology Modifiers

The compositions may optionally further comprise at least one thickening agent (also referred to as rheology or viscosity modifying agents). As non-limiting examples, polysaccharide-based thickeners, which are polymers having monosaccharides or disaccharides as base units, may be chosen. The polysaccharide thickeners which can be used in the compositions according to the present invention include, by way of example only, gums, celluloses, starches, or mixtures thereof.

Non-limiting examples of gums include acacia, agar, algin, alginic acid, ammonium alginate, amylopectin, calcium alginate, calcium carrageenan, carnitine, carrageenan, dextrin, gelatin, gellan gum, guar gum, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and biosaccharide gum. Modified gums or derivatives of gums may also be used, such as, for example, deacylated gellan gum, welan gum, or hydroxypropylated guar gum, such as Jaguar HP 105 sold by Rhodia.

Non-limiting examples of celluloses include hydroxyalkylcelluloses, such as hydroxyethylcellulose, hydroxypropylmethylcellulose, or hydropropylcelluloses, which may or may not contain a fatty chain. One particularly suitable hydroxypropylmethylcellulose is Methocel F4M sold by Dow Chemicals (INCI name: hydroxypropylmethylcellulose). Celluloses modified with groups comprising one or more nonionic fatty chains that can be used include hydroxyethylcelluloses, e.g. nonionic hydroxyethylcelluloses, modified by groups comprising at least one fatty chain, such as alkyl, arylalkyl or alkylaryl groups, or their mixtures, and in which the alkyl groups are preferably C8-C22 alkyl groups, such as the product NATROSOL™ Plus Grade 330 CS (C16 alkyls), sold by Aqualon, corresponding to the INCI name cetylhydroxyethylcellulose, or the product BERMOCOLL® EHM 100 sold by Berol Nobel, and those modified with alkylphenyl polyalkylene glycol ether groups, such as the product AMERCELL POLYMER® HM-1500 (nonylphenyl polyethylene glycol (15) ether) sold by Amerchol that corresponds to the INCI name nonoxynyl hydroxyethylcellulose.

Non-limiting examples of starches include modified starches, starch-based polymers, methylhydroxypropyl starch, potato starch, wheat starch, rice starch, starch crosslinked with octenyl succinic anhydride, starch oxide, dialdehyde starch, dextrin, British gum, acetyl starch, starch phosphate, carboxymethyl starch, hydroxyethyl starch, and hydroxypropyl starch.

If present, the total amount of thickening agents may vary, but typically ranges from about 0.01% to about 5%, including all ranges and subranges therebetween, such as from about 0.01% to about 4%, from about 0.01% to about 3%, from about 0.01% to about 2%, from about 0.01% to about 1.5%, from about 0.01% to about 1%, from about 0.01% to about 0.5%, from about 0.1% to about 5%, from about 0.1% to about 4%, from about 0.1% to about 3%, from about 0.1% to about 2%, from about 0.1% to about 1.5%, from about 0.1% to about 1%, or from about 0.1% to about 0.5%, by weight, relative to the total weight of the composition. In at least certain embodiments, the composition comprises a thickener in an amount less than about 1%, less than about 0.75%, less than about 0.5%, less than about 0.25%, or less than about 0.1% by weight, relative to the total weight of the composition.

### Solvents

Compositions according to the disclosure comprise at least one solvent. The solvent may be chosen from water, non-aqueous solvents, or mixtures thereof.

In some embodiments, the solvent comprises, consists essentially of, or consists of water. The total amount of water in the compositions may vary depending on the type of composition and the desired consistency, viscosity, etc. In various embodiments, water is present in the compositions in an amount ranging from about 60% to about 95%, such as about 65% to about 90%, about 70% to about 85%, or about 75% to about 85% by weight, relative to the total weight of the composition.

In certain embodiments, the composition comprises one or more non-aqueous solvents, for example, glycerin, C₁₋₄ alcohols, organic solvents, fatty alcohols, fatty ethers, fatty esters, polyols, glycols, vegetable oils, mineral oils, liposomes, laminar lipid materials, or any a mixture thereof. Non-limiting examples of solvents which may be used include alkanediols such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbitol, sorbitan, acetine, diacetine, triacetine, sulfolane, and a mixture thereof.

The total amount of solvent may range from about 60% to about 98% by weight, relative to the total weight of the composition, including all ranges and subranges therebetween. For example, in one embodiment, the total amount of solvent may be about 70% to about 95%, about 70% to about 90%, about 75% to about 85%, or about 75% to about 80% by weight, relative to the total weight of the composition.

### Auxiliary Components

Compositions according to the disclosure may optionally comprise any auxiliary component suitable for use in such compositions. Such components may include, but are not limited to, suspending agents such as carbomer and/or acrylates for suspending anti-dandruff agents, dyes/pigments, fillers, structuring agents, shine agents, antioxidants or reducing agents, penetrants, sequestrants, fragrances, buffers, dispersants, plant extracts, preserving agents, opacifiers, sunscreen agents, pH adjusting agents, and antistatic agents.

Optional auxiliary components may be present in the composition in a total amount ranging up to about 15%, such as up to about 10%, up to about 5%, up to about 3%, up to about 2%, or up to about 1% by weight, relative to the total weight of the composition. For example, compositions according to the disclosure may comprise a total amount of auxiliary components ranging from about 0.001% to about 5%, from about 0.005% to about 4%, from about 0.01% to about 3%, from about 0.05% to about 2.5%, or from about 0.1% to about 2% by weight, relative to the total weight of the composition.

In various embodiments, the compositions according to the present disclosure are stable, meaning that no phase separation or significant change in pH or viscosity is seen when stored at a temperature ranging from about 4° C. to about 45° C., such as from about 10° C. to about 37° C., or from about 20° C. to about 30° C., for at least about 8 weeks.

In at least some embodiments, compositions according to the disclosure may impart one or more properties such as smoothness, conditioning, excellent detangling, anti-frizz, ease of shaping and/or combing, anti-static, clean and/or smooth appearance to hair, and may impart one or more benefits to the scalp such as improved hydration, reduced dryness, reduced flaking, and/or reduced itching.

### II. METHODS

The disclosure also relates to methods for caring for and/or conditioning keratin materials, especially the hair and/or the scalp, with the compositions disclosed herein. The methods also relate to methods for conditioning the hair, methods for caring for the scalp, methods for treating dandruff, methods for reducing the appearance of dandruff, and methods for reducing symptoms of dandruff such as itching and flaking. Without limitation, methods according to the disclosure may include applying a sufficient amount, or an effective amount, of a composition disclosed herein to a keratin material, such as hair or scalp, which may be wet, damp, or dry, optionally allowing the composition to remain on the keratin material for a desired amount of time, and optionally rinsing the composition from the keratin material. In some embodiments, the composition is applied to the keratin materials as part of a personal care routine, in particular a hair and/or scalp care routine. For example, the composition may be applied to the hair and/or scalp after the hair and/or scalp has been cleansed with a shampoo composition, for example an anti-dandruff shampoo composition.

In certain embodiments, compositions according to the disclosure are particularly useful for providing one or more desirable cosmetic benefits to the hair, for example, shine, smoothness, detangling, and/or anti-frizz properties. In addition, in certain embodiments, compositions according to the disclosure are particularly useful for caring for the scalp, for example for providing moisture, hydration, and/or anti-dandruff benefits to the scalp. Accordingly, the disclosure encompasses methods for caring for, treating, and/or conditioning the hair and/or scalp with compositions according to the disclosure.

Such methods may, for example, include applying an effective amount of a composition of the instant disclosure to the hair, allowing the composition to remain on the hair for a period of time, and subsequently rinsing the composition from the hair, followed by allowing the hair to air dry or drying the hair with a hair dryer which blows air through the hair and accelerates drying.

Usually, the composition is allowed to remain on the hair for at least a period of time sufficient to incorporate the composition throughout the hair, for example, by using one's hands. However, the amount of time may vary and may be any amount of time useful to permit the various components of the composition to interact with the hair and/or scalp. Thus, by way of example only, the composition may be allowed to remain on the hair for a leave-in period of up to about 30 minutes, such as from about 5 seconds to about 15 minutes, about 5 seconds to about 10 minutes, about 5 seconds to about 5 minutes, about 10 seconds to about 5 minutes, or about 10 seconds to about 3 minutes. Alternatively, the composition may be removed substantially immediately after it is applied, e.g. without any leave-in period, or may be left on the hair for a longer period such as overnight.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense.

As used herein, the use of the singular includes the plural unless specifically stated otherwise. The singular forms “a,” “an,” “the,” and “at least one” are understood to encompass the plural as well as the singular unless the context clearly dictates otherwise. The expression “one or more” and “at least one” are interchangeable and expressly include individual components as well as mixtures/combinations. Likewise, the term “a salt thereof” also relates to “salts thereof.” Thus, where the disclosure refers to “at least one element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two or more of A, B, C, D, E, F, one or more salts of A, one or more salts of B, one or more salts of C, one or more salts of D, one or more salts of E, and one or more salts of F may be included.

The term “and/or” should be understood to include both the conjunctive and the disjunctive. For example, “water and/or non-aqueous solvents” means “water and non-aqueous solvents” as well as “water or non-aqueous solvents,” and expressly covers instances of either.

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

All ranges and amounts given herein are intended to include sub-ranges and amounts using any disclosed point as an end point, and all endpoints are intended to be included unless expressly stated otherwise. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not expressly stated, unless expressly stated otherwise. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Unless expressly stated otherwise, “about” means +/−5%. Likewise, all endpoints of ranges are understood to be individually disclosed, such that, for example, a range of 1:2 to 2:1 is understood to disclose a ratio of both 1:2 and 2:1.

All amounts and ratios herein are given by weight, based upon the total weight of the composition, unless otherwise indicated. Unless otherwise indicated, all percentages herein are by weight of active material.

As used herein, the term “salts” referred to throughout the disclosure may include salts having a counterion such as an alkali metal, alkaline earth metal, or ammonium counterion. This list of counterions, however, is non-limiting. Salts also include a dissociated form of a compound, e.g. in an aqueous solution.

As used herein, the terms “applying a composition onto keratin materials,” “applying a composition onto hair,” and variations of these phrases are intended to mean contacting the keratin material or hair with at least one of the compositions according to the disclosure, in any manner.

As used herein, the terms “treating dandruff,” “reducing the appearance of dandruff,” or “reducing the symptoms of dandruff” mean any reduction in the number of or degree of symptoms of dandruff, such as flakes and/or itchiness of the scalp.

“Cosmetically acceptable” means compatible with keratinous materials. For example, “cosmetically acceptable carrier” means a carrier that is compatible with any keratinous tissue.

“Keratin material” as used herein includes skin and hair, such as hair on the human head.

As used herein, the term “conditioning” means imparting to keratin materials at least one property chosen from increased ease of combability, anti-frizz, manageability, luster, shine, and softness. The state of conditioning can be evaluated by any means commonly used in the art, such as, for example, measuring, and comparing, the ease of combability of the treated hair and of the untreated hair in terms of combing work, or by considering individual perception, for example perception of the consumer or hair stylist.

As used herein, the term “treating” (and its grammatical variations) keratin materials refers to the application of the compositions of the disclosure onto the surface of keratin materials, such as hair and the scalp.

Unless otherwise defined for any specific embodiment, the term “substantially free” and “essentially free,” which are used interchangeably herein, mean that there is less than about 2% by weight of a specific material added to a composition, based on the total weight of the composition. For example, the compositions may include less than about 1.5%, less than about 1%, less than about 0.5%, less than about 0.1%, less than about 0.01%, less than about 0.001%, or less than about 0.0001% of the specified material. As such, it is contemplated that any component described herein for use in the compositions can be present in the compositions in amounts less than about 2%, less than about 1.5%, less than about 1%, less than about 0.5%, less than about 0.1%, less than about 0.01%, less than about 0.001%, or less than about 0.0001%, and the composition will be considered “substantially free” or “essentially free” of such material. A composition that is “free” of a component is understood to contain none of the specified component. However, it is understood that the terms “free” and “substantially free” refer to the amount of a component added to the composition, without including an amount of the component present in the composition as a minor component in a raw material. For example, a composition that is “free” of waxes may not have wax included as an intended component but may nevertheless contain a pigment that is coated with a wax, as such wax would be considered a minor component of the pigment material and would not be expected to provide benefits to the composition that would be expected by including a wax per se as an intended component.

“Substituted,” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

Unless otherwise expressly stated, it is in no way intended that any method set forth herein be construed as requiring that its steps be performed in a specific order. Accordingly, where a method does not expressly recite that a particular order of steps must be followed or it is not otherwise specifically stated that the steps are to be limited to a specific order, it is no way intended that any particular order be inferred.

It should also be understood that the precise numerical values used in the specification and claims form additional embodiments of the disclosure and are intended to include any ranges which may be narrowed to any two end points disclosed within the exemplary ranges and values provided, as well as the specific end points themselves.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The examples that follow serve to illustrate embodiments of the disclosure without, however, being limiting in nature.

### EXAMPLES

The following examples are intended to be non-limiting and explanatory in nature only. In the Examples, amounts are expressed in percentage by weight (wt %) of active materials, relative to the total weight of the composition.

### Example 1—Compositions

Compositions 1A-1L according to the disclosure were prepared as set forth in Tables 1A-1 and 1A-2.

TABLE 1A-1

| | 1A | 1B | 1C | 1D | 1E | 1F |
|---|---|---|---|---|---|---|
| CERAMIDES | 0.0039 | 0.0039 | | | 0.4 | 0.3 |
| PHYTOSPHINGOSINE | 0.0014 | 0.0014 | | | 0.0014 | 0.0014 |
| CHOLESTEROL | 0.0013 | 0.0013 | | | 0.0013 | 0.0013 |
| VITAMINS | 0.1 | 0.1 | 0.1 | 0.1 | 0.1 | 0.1 |
| CATIONIC SURFACTANTS | 4.0 | 4.0 | 4.0 | 4.0 | 4.0 | 4.0 |
| ZINC PYRITHIONE | 0.5 | | 0.5 | 0.5 | 0.5 | 0.5 |
| MOISTURIZING AGENTS | 4.0 | 4.0 | 4.0 | 4.0 | 4.0 | 4.0 |
| SODIUM HYALURONATE | 0.01 | 0.01 | 0.5 | 0.75 | 0.25 | 0.1 |
| SODIUM LAUROYL | 0.03 | 0.03 | 0.03 | 0.03 | 0.03 | 0.03 |
| LACTYLATE | | | | | | |
| CETYL ESTERS | 1.0 | 1.0 | 1.0 | 1.0 | 1.0 | 1.0 |
| TRIDECETH-6 | 0.09 | 0.09 | 0.09 | 0.09 | 0.09 | 0.09 |
| CARBOMER | 0.0008 | 0.0008 | 0.0008 | 0.0008 | 0.0008 | 0.0008 |
| CETEARYL ALCOHOL | 5.2 | 5.2 | 5.2 | 5.2 | 5.2 | 5.2 |
| AMODIMETHICONE | 1.0 | 1.0 | 1.0 | 1.0 | 1.0 | 1.0 |
| FATTY ACIDS | | | | 0.75 | 0.25 | |
| ADDITIVES* | <3 | <3 | <3 | <3 | <3 | <3 |
| SOLVENTS (water and non- | QS | QS | QS | QS | QS | QS |
| aqueous solvents) | to 100 | to 100 | to 100 | to 100 | to 100 | to 100 |

*preservatives, thickeners, pH adjusters, stabilizers, antioxidants

TABLE 1A-2

| | 1G | 1H | 1I | 1J | 1K | 1L |
|---|---|---|---|---|---|---|
| CERAMIDES | 0.5 | 0.75 | 1.0 | 0.0039 | 0.0039 | |
| PHYTOSPHINGOSINE | 0.0014 | 0.0014 | 0.0014 | 0.0014 | 0.0014 | |
| CHOLESTEROL | 0.0013 | 0.0013 | 0.0013 | 0.0013 | 0.0013 | |
| VITAMINS | 0.1 | 0.1 | 0.1 | 0.1 | 0.1 | 0.1 |
| CATIONIC SURFACTANTS | 4.0 | 4.0 | 4.0 | 4.0 | 4.0 | 4.0 |
| ZINC PYRITHIONE | 0.5 | 0.5 | 0.5 | 0.5 | 0.5 | 0.5 |
| MOISTURIZING AGENTS | 4.0 | 4.0 | 4.0 | 4.0 | 4.0 | 4.0 |
| SODIUM HYALURONATE | 0.01 | 0.01 | 0.01 | 0.01 | 0.01 | |
| SODIUM LAUROYL | 0.03 | 0.03 | 0.03 | 0.03 | 0.03 | 0.03 |
| LACTYLATE | | | | | | |
| CETYL ESTERS | 1.0 | 1.0 | 1.0 | 1.0 | 1.0 | 1.0 |
| TRIDECETH-6 | 0.09 | 0.09 | 0.09 | 0.09 | 0.09 | 0.09 |
| CARBOMER | 0.0008 | 0.0008 | 0.0008 | 0.0008 | 0.0008 | 0.0008 |
| CETEARYL ALCOHOL | 5.2 | 5.2 | 5.2 | 5.2 | 5.2 | 5.2 |
| AMODIMETHICONE | 1.0 | 1.0 | 1.0 | 1.0 | 1.0 | 1.0 |
| ADDITIVES* | <3 | <3 | <3 | <3 | <3 | <3 |
| SOLVENTS (water and non- | QS | QS | QS | QS | QS | QS |
| aqueous solvents) | to 100 | to 100 | to 100 | to 100 | to 100 | to 100 |

*preservatives, thickeners, pH adjusters, stabilizers, antioxidants

The compositions shown in Tables 1A-1 and 1A-2 were prepared with the specified amounts of ceramides (ceramide AP, ceramide NP, and/or ceramide EOP), vitamins (tocopheryl acetate and/or niacinamide), cationic surfactants (cetrimonium chloride, behentrimonium chloride, and/or behentrimonium methosulfate), moisturizing agents other than sodium hyaluronate (caprylyl glycol, PEG-180, glycerin, and/or propylene glycol), and fatty acids (linoleic acid and α-linolenic acid), where present. Compositions 1A-1L were stable, even with the benefit agents and anti-dandruff active agents present.

A shampoo composition (SHMP) as shown in Table 1B was prepared.

TABLE 1B

| | SHMP |
|---|---|
| CERAMIDES | 0.0039 |
| CATIONIC SURFACTANT | 0.08 |
| ZINC PYRITHIONE | 1.02 |
| SODIUM LAURYL SULFOACETATE | 1.05 |
| ACRYLATES COPOLYMER | 0.42 |
| DISODIUM LAURETH SULFOSUCCINATE | 2.7 |
| PHYTOSPHINGOSINE AND/OR CHOLESTEROL | 0.0027 |
| CETEARYL ALCOHOL | 0.1875 |
| COCAMIDE MIPA | 0.7 |
| COCO-BETAINE | 0.27 |
| SODIUM C14-16 OLEFIN SULFONATE | 4.56 |
| AMODIMETHICONE | 1.14 |
| POLYQUATERNIUM-10 | 0.8 |
| DISODIUM COCOAMPHODIACETATE | 2.52 |
| TRIETHYL CITRATE | 0.0017 |
| TRIDECETH-6 | 0.1 |
| CARBOMER | 0.35 |
| SODIUM HYALURONATE | 0.01 |
| PPG-5-CETETH-20 | 0.5 |
| GLYCOL DISTEARATE | 1.35 |
| ADDITIVES* | <3 |
| SOLVENTS (water and non-aqueous solvents) | QS to 100 |

*preservatives, vitamins, pH adjusters, stabilizers, consistency agents, electrolytes, antioxidants, emulsifiers

Table 1C shows commercially-available anti-dandruff shampoo and conditioner compositions with the ingredients as listed on the package.

TABLE 1C

| PRODUCT | INGREDIENTS LISTED ON PACKAGE |
|---|---|
| Comparative | Active Ingredient: Pyrithione Zinc 1%, Water, Sodium Lauryl |
| Shampoo #1 | Sulfate, Sodium Laureth Sulfate, Glycol Distearate, Sodium |
| C1-S | Chloride, Zinc Carbonate, Fragrance, Cocamidopropyl Betaine, |
| | Sodium Xylenesulfonate, Dimethicone, Sodium Benzoate, Guar |
| | Hydroxypropyltrimonium Chloride, Magnesium Carbonate |
| | Hydroxide, Blue 1, Red 33 |
| Comparative | Pyrithione Zinc 0.5%, Water, Stearyl Alcohol, Cetyl Alcohol, |
| Conditioner | Stearamidopropyl Dimethylamine, Glutamic Acid, Dimethicone, |
| C1-C | Fragrance, Phenoxyethanol, Benzyl Alcohol, Citric Acid, Sodium |
| | Chloride, Prunus Amygdalus Dulcis (Sweet Almond) Oil, |
| | Methylchloroisothiazolinone, Methylisothiazolinone |

### Example 2—Comparison of Properties of Compositions and Treated Hair/Scalp

In order to evaluate the properties of compositions according to the disclosure and hair treated with the compositions, the following study was performed. In a salon setting, equal amounts of one of compositions SHMP (side A) or C1-S (side B) were applied to opposite sides of the head of three human volunteers and massaged into the hair for 15 seconds, after which the hair was rinsed with water for 15 seconds. Next, equal amounts of either composition 1A (side A) or C1-C (side B) were applied to the volunteers' hair and left to rest under ambient conditions for three minutes, and the hair was then thoroughly rinsed before being dried with a blow dryer. Properties of each composition and properties imparted to the hair by each composition were evaluated by an expert, as follows.

### Example 2-1—Properties of Compositions

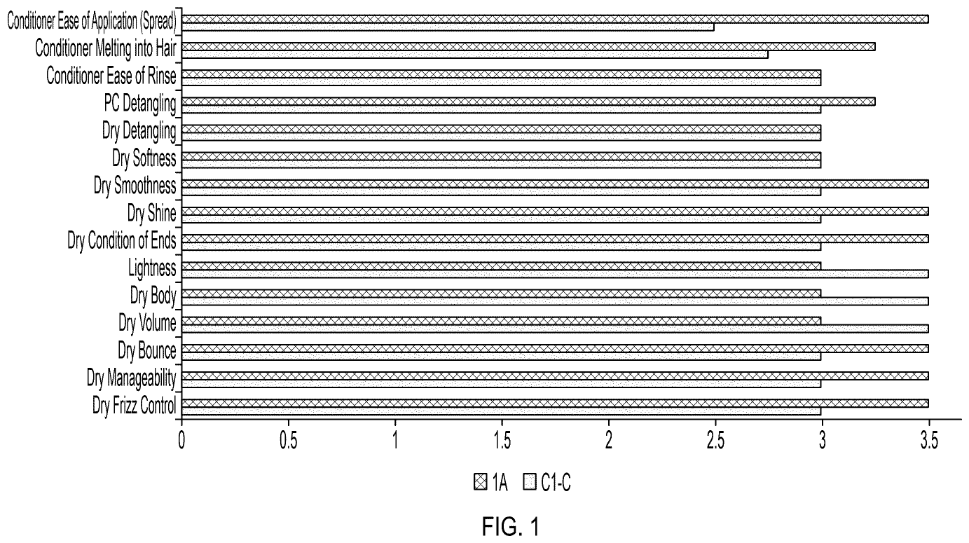

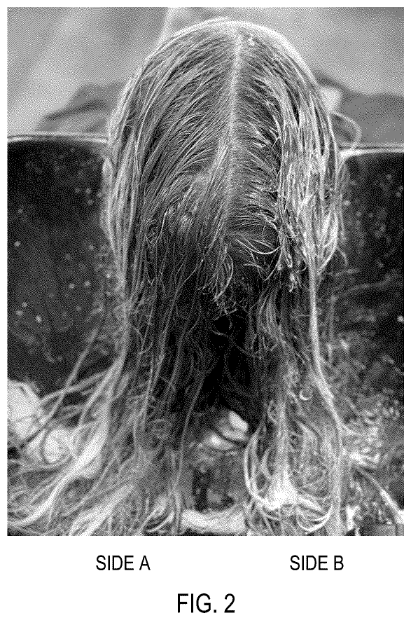

During the routine, the expert evaluated properties of compositions 1A or C1-C. As the data in FIG. 1 show, composition 1A according to the disclosure scored significantly higher than composition C1-C in both ease of application and melting into the hair. The expert noted that, as shown in FIG. 2, comparative composition C1-C remained on the surface of the hair (side B), whereas composition 1A according to the disclosure (side A) was easily absorbed into the hair.

### Example 2-2—Properties of Treated Hair and Scalp

Once compositions 1A or C1-C were rinsed from the hair, the expert evaluated the ease of wet, post-conditioning (PC) detangling of the treated hair. As FIG. 1 shows, composition 1A provided significantly greater ease of wet detangling of the hair than composition C1-C.

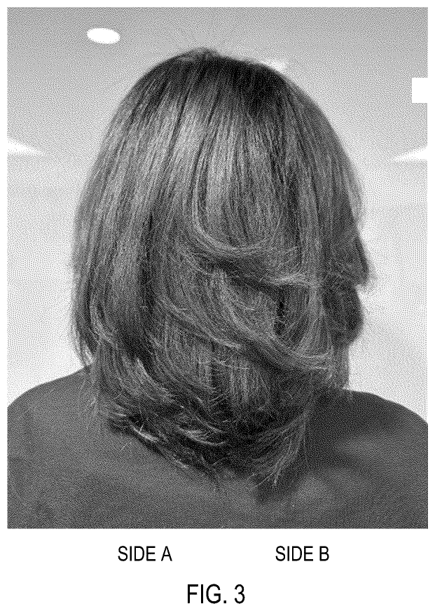

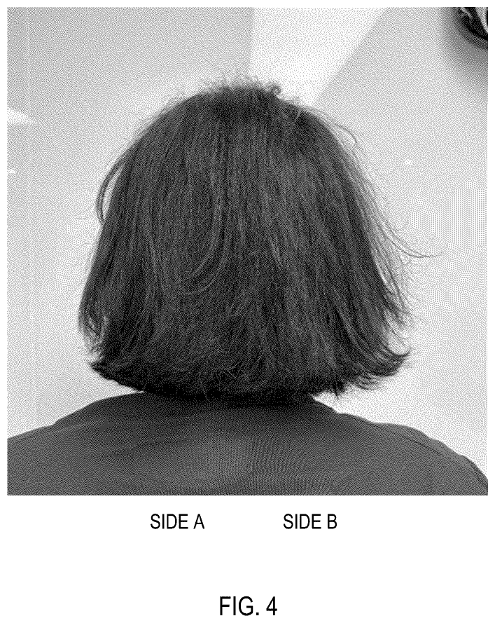

After the hair was dried, the expert evaluated dry properties of both sides of the volunteers' hair. As FIG. 1 shows, the hair treated with composition 1A according to the disclosure had significantly better smoothness, shine, end properties (reduced appearance of split ends), bounce, manageability, and frizz control than hair treated with composition C1-C. FIGS. 3 and 4 show images of the dried hair, where the expert noted that the hair on side A of FIG. 3 was consistently smooth from root to ends with less frizz and appeared less heavy or weighed down than side B, and the hair on side A of FIG. 4 felt smooth and was shiny with less frizz than the hair on side B which felt dry and appeared frizzy and unruly.

The results showed that the treated hair was much more manageable, soft, and shiny after treatment with shampoo SHMP and composition 1A than after treatment with shampoo C1-S and conditioner C1-C. The results also demonstrated that the scalp of the volunteers appeared healthier, more hydrated, and cleaner after treatment with shampoo SHMP and composition 1A than after treatment with shampoo C1-S and conditioner C1-C, demonstrating that composition 1A improved the scalp care with the use of benefit agents in both the shampoo and conditioner.

### Example 3—Additional Evaluations

The procedure of Example 2 was repeated on ten (10) separate swatches of Type 4 hair, using composition SHMP from Table 1B and one of compositions 1C-1L from Tables 1A-1 and 1A-2. Once dry, the properties of the treated swatches were evaluated, with the results being the same as seen with composition 1A in Example 2. The expert observed that increasing the amount of benefit agent improved the overall smoothness and frizz control, with the swatch treated with composition 1I having the best softness, shine, and anti-frizz properties. The expert also observed that the swatches treated with compositions including ceramides had the best anti-frizz benefits.

The above Examples, which are considered to be statistically significant in terms of both observed improvement in properties of the compositions and treated hair as well as repeatability, demonstrate that stable compositions containing benefit agents and/or anti-dandruff active agents, which have desirable composition and treatment properties, can be prepared.

### Example 4—Additional Compositions

Compositions 2A-2D can also be prepared as shown in Table 2, and are likewise expected to have similar properties and provide similar benefits to the hair as compositions 1A-1L.

TABLE 2

| | 2A | 2B | 2C | 2D |
|---|---|---|---|---|
| CERAMIDES | 1.5 | 0.1 | | |
| PHYTOSPHINGOSINE | 0.05 | 0.1 | | 0.3 |
| CHOLESTEROL | | | 0.1 | 0.1 |
| VITAMIN B3 | 0.1 | | | 0.5 |
| CATIONIC | 5.0 | 2.5 | 1.0 | 0.7 |
| SURFACTANTS | | | | |
| ZINC PYRITHIONE | 1.5 | | | 0.15 |
| SALICYLIC ACID | | 0.5 | 0.5 | |
| BUTYLENE GLYCOL | 2.5 | 1.0 | 5.0 | |
| SODIUM | | 0.01 | | 0.75 |
| HYALURONATE | | | | |
| CETEARYL ALCOHOL | 1.0 | 1.0 | 1.5 | |
| AMODIMETHICONE | | 1.0 | | |
| STEARIC ACID | | | 0.08 | |
| CAPRIC ACID | | 1.0 | | 0.50 |
| ADDITIVES | <3 | <3 | <3 | <3 |
| SOLVENTS | QS to 100 | QS to 100 | QS to 100 | QS to 100 |

The above Examples demonstrate that hair conditioner compositions that are stable and provide excellent scalp care, while also providing excellent cosmetic properties to hair, can be achieved. These results were surprising because conditioner compositions are not used for scalp care.

1. A hair conditioner composition comprising:
(a) at least one cationic surfactant;
(b) at least one ceramide;
(c) optionally, at least one anti-dandruff active agent; and
(d) water,
wherein the total amount of cationic surfactants ranges from about 0.1% to about 15% by weight, relative to the total weight of the composition, and
wherein the total amount of benefit agents ranges from about 0.0001% to about 3% by weight, relative to the total weight of the composition.

2. The hair conditioner composition of claim 1, wherein the composition comprises at least one ceramide and the total amount of ceramides ranges from about 0.0005% to about 3% by weight, relative to the total weight of the composition.

3. The hair conditioner composition of claim 2, comprising ceramide AP, ceramide NP, ceramide EOP, or combinations thereof.

4. The hair conditioner composition of claim 1, comprising at least one anti-dandruff active agent.

5. The hair conditioner composition of claim 4, wherein the total amount of anti-dandruff active agents ranges from about 0.01% to about 10% by weight, relative to the total weight of the composition.

6. The hair conditioner composition of claim 4, wherein the at least one anti-dandruff active agent is chosen from ellagic acid, ethers of ellagic acid, salts of ellagic acid, salts of ethers of ellagic acid, pyrithione, salts of pyrithione, selenium (poly)sulfides, salicylic acid, piroctone olamine, tea tree oil, climbazole, fluocinolone, ketoconazole, coal tar, or combinations thereof.

7. The hair conditioner composition of claim 1, further comprising at least one fatty acid chosen from saturated or unsaturated fatty acids having from 8 to 30 carbon atoms.

8. The hair conditioner composition of claim 7, comprising at least one fatty acid chosen from unsaturated ω-3 fatty acids having from 16 to 24 carbon atoms, unsaturated ω-6 fatty acids having from 16 to 24 carbon atoms, or combinations thereof.

9. The hair conditioner composition of claim 7, comprising at least one fatty acid chosen from oleic acid, lauric acid, palmitic acid, myristic acid, stearic acid, linoleic acid, capric acid, caprylic acid, α-linolenic acid, arachidonic acid, eicosapentaenoic acid, docosahexaenoic acid, or combinations thereof.

10. The hair conditioner composition of claim 7, wherein the total amount of fatty acids present in the composition ranges from about 0.001% to about 3% by weight, relative to the total weight of the composition.

11. The hair conditioner composition of claim 1, comprising at least one ceramide and at least one additional benefit agent chosen from phytosphingosine and cholesterol.

12. The hair conditioner composition of claim 1, further comprising at least one moisturizing agent.

13. The hair conditioner composition of claim 1, further comprising at least one hair conditioning agent.

14. A composition for caring for keratin materials comprising:
(a) at least one cationic surfactant;
(b) at least one ceramide;
(c) at least one anti-dandruff active agent;
(d) water; and
(e) at least one additional component chosen from fatty acids, moisturizing agents, hair conditioning agents, thickening agents, or combinations thereof,
wherein the total amount of cationic surfactants ranges from about 1% to about 10% by weight, relative to the total weight of the composition,
wherein the total amount of ceramides ranges from about 0.0001% to about 3% by weight, relative to the total weight of the composition, and
wherein the composition comprises less than about 3% of cleansing surfactants.

15. The composition of claim 14, comprising at least one ceramide chosen from ceramide AP, ceramide NP, ceramide EOP, or combinations thereof.

16. The composition of claim 15, comprising at least one anti-dandruff active agent chosen from ellagic acid, ethers of ellagic acid, salts of ellagic acid, salts of ethers of ellagic acid, pyrithione, salts of pyrithione, selenium (poly)sulfides, salicylic acid, piroctone olamine, tea tree oil, climbazole, fluocinolone, ketoconazole, coal tar, or combinations thereof.

17. The composition of claim 16, comprising at least one fatty acid chosen from oleic acid, lauric acid, palmitic acid, myristic acid, stearic acid, linoleic acid, capric acid, caprylic acid, α-linolenic acid, arachidonic acid, eicosapentaenoic acid, docosahexaenoic acid, or combinations thereof.

18. The composition of claim 17, comprising at least one moisturizing agent and/or at least one hair conditioning agent.

19. A method for caring for keratin materials comprising applying a composition to the keratin materials, the composition comprising:
(a) at least one cationic surfactant;
(b) at least one benefit agent chosen from ceramides, sterols, phytosphingosine, or combinations thereof;
(c) optionally, at least one anti-dandruff active agent; and
(d) water,
wherein the total amount of cationic surfactants ranges from about 0.1% to about 15% by weight, relative to the total weight of the composition, and
wherein the total amount of benefit agents ranges from about 0.0001% to about 3% by weight, relative to the total weight of the composition.

20. The method of claim 19, further comprising a step of rinsing the composition from the keratin materials.
